(19) United States
(12) Patent Application Publication
(10) Pub. No.: US 2021/0005320 A1
(43) Pub. Date: Jan. 7, 2021

(54) METHOD OF INFERRING A NEED FOR MEDICAL TEST

(71) Applicant: Combinostics Oy, Tampere (FI)

(72) Inventors: Jyrki LÖTJÖNEN, Tampere (FI); Juha KOIKKALAINEN, Tampere (FI)

(73) Assignee: Combinostics Oy, Tampere (FI)

(21) Appl. No.: 16/917,349

(22) Filed: Jun. 30, 2020

(30) Foreign Application Priority Data
Jul. 2, 2019 (FI) 20195603

(51) Int. Cl. G16H 50/20; G16H 50/50; G16H 50/70; G16H 70/60; G16H 30/20; G16H 30/40; G16H 10/20; G16H 20/10; G16H 40/20

(52) U.S. Cl. CPC G16H 50/20; G16H 50/50; G16H 50/70; G16H 70/60; G16H 50/30; G16H 30/40; G16H 10/20; G16H 20/10; G16H 40/20; G16H 30/20

(57) ABSTRACT

A method for inferring a justification for a further medical test, including: calculating a first value of confidence of medical decision by comparing data from a subject with corresponding data of subjects from a reference database, if the first value of confidence of medical decision is smaller than a first cutoff, which defines the minimum acceptable confidence value for making a medical decision, the following steps are performed: simulating at least one value indicative on one medical condition for the further medical test, wherein said at least one value represents typical value for said medical condition, and augmenting the data from a subject with said at least one simulated value from said one medical condition, and calculating a second value of confidence of medical decision by comparing said augmented data from a subject with corresponding data of subjects from the reference database. The disclosed embodiments further relate to a computer program product and device performing the method.

### TECHNICAL FIELD

The aspects of the disclosed embodiments relate to a method of inferring a justification for performing a further medical test. The aspects of the disclosed embodiments also relate to an apparatus, and a computer program product for inferring a justification for performing a further medical test.

### BACKGROUND

Medical decisions are often based on patient data from multiple medical tests. A medical need defines typically which test or tests are performed but availability, medical risks and costs may also impact the decision. Specialists diagnosing patients define the need of such tests based on their own expertise and/or following guidelines of their hospital. It is likely that several patients get additional tests although they do not necessarily benefit from them and several patients do not get additional tests although they would be useful for making an accurate medical decision, such as diagnosis or treatment decision.

Therefore, there is a need for a systematic and data-driven approach for helping medical decision makers to enter to a more justified decision about whether a certain further test is needed and should be performed.

### SUMMARY

Now there has been invented an improved method and technical equipment implementing the method, by which the above problems are alleviated. Various aspects of the disclosed embodiments include a method, an apparatus, and a computer readable medium comprising a computer program stored therein, which are characterized by what is stated in the independent claims. Various embodiments are disclosed in the dependent claims.

The aspects of the disclosed embodiments relate to a method, apparatus, system, and computer program for inferring a justification for performing a further medical test. In other ways, the idea is to determine whether the further medical test would provide such information that it is possible confidently enough make a medical decision. According to a first aspect, there is provided a method for inferring a justification for a further medical test, comprising calculating a first value of confidence of medical decision by comparing data from a subject with corresponding data of subjects from a reference database, and if the first value of confidence of medical decision is smaller than a first cutoff, which defines the minimum acceptable confidence value for making a medical decision, the following steps are performed: simulating at least one value indicative on one medical condition for the further medical test, wherein said at least one value represents typical value for said medical condition, augmenting the data from a subject with said at least one simulated value from said one medical condition, calculating a second value of confidence of medical decision by comparing said augmented data from a subject with corresponding data of subjects from the reference database, and indicating that the further medical test is justified if said second value of confidence of medical decision is higher than a second cutoff.

According to an example, a value of confidence of medical decision is defined using a probabilistic measure. According to an example, a value of confidence of medical decision is defined using a probability of correct class (PCC). According to an example, a value of confidence of medical decision is defined using disease-state index, which measures the location of said data from a subject relative to two groups of subjects. According to an example, said data from a subject comprises medical test data. According to an example, said data comprises data from cognitive tests or magnetic resonance imaging. According to an example, said data from a subject comprises background factors of the subject. According to an example background factors of the subject comprise age, gender, number of education years, information about co-morbidities, or medications used. According to an example said at least one simulated value of the further medical test is corrected for at least one background factor of the subject. According to an example, said at least one simulated value of the further medical test is corrected for medical test data of the subject. According to an example, the further medical test is cerebrospinal fluid biomarkers or positron emission tomography imaging. According to an example, said confidence of medical decision is confidence of giving a certain treatment. According to an example, said confidence of medical decision is confidence of diagnosis. According to an example, said confidence of medical decision is confidence of diagnosis in cognitive disorders. According to an example, the first cutoff is the same as the second cutoff.

According to a second aspect, there is provided a computer program product embodied on a non-transitory computer readable medium. The computer program product comprises computer instructions that, when executed on at least one processor of a system or an apparatus, is configured to perform the method for inferring a justification for a further medical test according to the first aspect and its examples.

According to a third aspect, there is provided a device for inferring a justification for a further medical test. The device comprises means for performing the method for inferring a need for a further medical test according to the first aspect and its examples.

### BRIEF DESCRIPTION OF THE DRAWINGS

In the following, various embodiments of the present disclosure will be described in more detail with reference to the appended drawings, in which

FIG. 1 shows, by way of example, a method for inferring a justification for performing a further medical test;

FIG. 2a shows, by way of example, a system arranged to infer and/or display a justification for a further medical test;

FIG. 2b shows, by way of example, devices arranged to infer and/or display a justification for a further medical test;

FIG. 3 shows, by way of example, a representation and visualization of medical test results with and without simulated further data from a subject; and

FIG. 4 shows, by way of example, a method for inferring a justification for performing a further medical test.

### DETAILED DESCRIPTION

Medical decisions, like decision of a diagnosis, of performing a surgical operation or of starting a certain therapy, are often based on patient i.e. subject data obtained from performed medical test(s). A medical need defines typically which test or tests need to be or are justified to be performed, but availability, medical risks and costs of tests may also impact the decision. For example, when diagnosing cognitive disorders, basic cognitive tests, such as mini-mental state examination (MMSE), Montreal cognitive assessment (MOCA), Rey Auditory Verbal Learning Test (RAVLT) and/or Consortium to establish a registry for Alzheimer's disease (CERAD) tests, and anatomic imaging, such as magnetic resonance imaging (MRI) or computerized tomography (CT) imaging, are performed first. If the diagnosis cannot be established using these data, further tests, such as comprehensive neuropsychological tests, cerebrospinal fluid (CSF) biomarkers or positron emission tomography (PET), either FDG-PET or amyloid-PET, are requested. The additional cost of these tests and invasiveness of CSF sampling and PET imaging limit, however, the use of these tests although their use might be justified for medical reasons and they would likely bring clarity to diagnostics. If specialists diagnosing patients define the justification of such tests based on their own expertise and/or following guidelines of their hospital, it is likely that several patients get additional tests although they do not necessarily benefit from them and several patients do not get additional tests although they would be useful for making accurate diagnosis or treatment decisions. Unnecessary additional i.e. further tests not only cause needless costs, but they may reserve research resources from those patients who would have needed these tests and they may also have some unwanted effects on patients, for example increased risk of infection in a case of invasive tests. Therefore, a systematic and data-driven approach of the present disclosure that helps medical decision makers to make more justified decisions about whether a certain further test is justified and should be performed does not only decrease the number of unnecessary tests and costs, but it also guides specialists to choose a test or tests that more likely provides the best information about medical status of a patient. When a specialist makes a medical decision and considers whether a certain test or tests are justified, different background factors of a patient i.e. subject in question, such as age, gender, number of education years, information about co-morbidities and medications may also be used. For example, if a patient with cognitive problems is 20 years old, it is very unlikely that the cognitive decline is due to Alzheimer's disease. Thus, data from a patient that may also be called as data from a subject may comprise data that is obtained from performed medical test or tests i.e. medical test data and different patient's background factors.

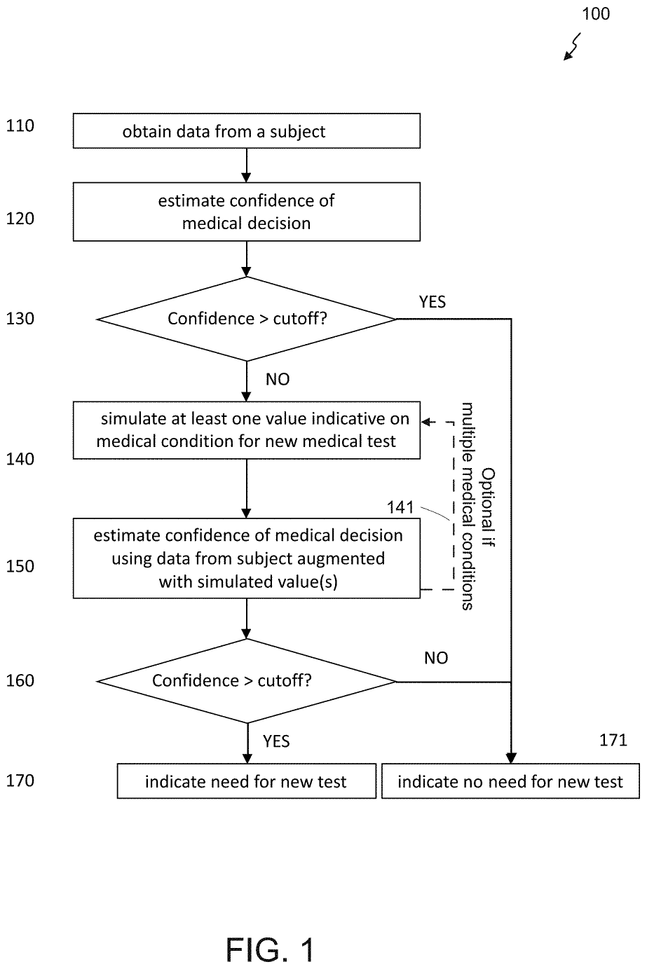

FIG. 1 shows, by way of example, a flow-chart of an inferring method 100 defining a justification for performing a further medical test in addition to existing, already performed medical test or tests. Differential diagnosis of cognitive disorders is used herein as an example to explain the aspects of the disclosed embodiments, but the inferring method can be used in any other medical decision-making task where the justification of performing a new further test is considered. For example, the inferring method may be applied in diagnostics or prognostics of different diseases or injuries, or predicting a disease or making treatment decisions, such as whether a patient requires a surgical operation or whether a certain therapy is applied, or deciding a need for monitoring such as whether a patient requires monitoring in intensive care unit. In addition to cognitive disorders, the aspects of the disclosed embodiments may be applied to many other areas of medicine, such as neurodegenerative diseases, neurology, internal medicine, oncology, paediatrics, or psychiatry.

An initial situation before starting the inferring method may be that a specialist considers whether additional testing is needed i.e. justified or whether she/he is confident enough for making a decision based on existing data of a subject comprising data obtained from a certain test or a set of tests performed for the patient, for example, medical test or set of medical tests and/or patient's background factors. These data may be obtained, for example, from a patient folder or database(s). In diagnosing cognitive disorders, the MMSE and CERAD cognitive tests and MRI imaging may have already been performed for the patient, and the specialist considers whether the patient has Alzheimer's disease based on the data of already made tests. If the specialist is confident that the patient has the disease, the diagnosis is given, and no additional testing is performed. If the specialist is not confident, she/he may order additional CSF biomarkers or amyloid-PET images and in a case of cancer she/he may order a biopsy. And, for example, when diagnosing cancer instead of cognitive disorders, this could mean that blood test and ultrasound imaging have been performed for a patient, and the specialist considers whether the patient has breast cancer based on the data of already made tests. If the specialist is confident that the patient does not have the cancer, the decision is given, and no additional testing is performed. If the specialist is not confident, she/he may order a biopsy. The specialist often needs to consider also the costs of these additional tests when doing the decision. Still today, specialists perform all this reasoning typically in their minds requiring strong expertise and being subjective. This inferring method of justification of a further test of the present disclosure may thus support specialists in making this reasoning more systematic and objective by giving information what effect the further test would have on the initial uncertainty or does it have any. There may not be single correct way to measure the confidence quantitatively. Multiple methods may be used but only a few examples are given here.

In step 110, data already existing and available for the patient is obtained. For example, in cognitive disorders the data may comprise the following kinds of data MMSE, CERAD and quantified measures from MRI images, such as the hippocampus volume or visually rated medial temporal lobe atrophy (MTA). In step 120, based on the obtained data, value of confidence of making a medical decision i.e. measure about confidence provided by medical test, is estimated, for example by calculating. In traditional implementation, a specialist could define a set of rules based on cutoff i.e. threshold values for these tests results from the literature. If all data pointed to Alzheimer's disease, the confidence could be regarded high and the specialist might give a diagnosis. However, this traditional implementation approach simplifies the challenge considerably: 1) cognitive disorders may be caused by a high number of different diseases and other indications with overlapping symptoms, 2) cognitive diseases are often progressive meaning that patients go through all stages from mild to severe making data interpretation much more challenging, 3) the patient's background factors, such as age and education, may affect how the test results should be interpreted, 4) the set of test results available is often more comprehensive than described above, and 5) non-quantitative information from interviewing the patient and care givers also impact the decision. Because of these points 1 to 5, a more systematic framework to measure the status of the patient and the confidence of making a medical decision are performed in further steps of the method 100.

Disease-state index (DSI) is an example of calculating a value of confidence of making a medical decision (step 120). DSI a technology for measuring the state or the “location” of the patient relative to two diagnostic groups, for example between healthy controls and Alzheimer's disease patients. DSI is composed of two components: fitness and relevance. Fitness measures how similar a certain test result of the patient is to the results of the same test from previous patients belonging to two diagnostics groups, for example healthy controls (negative group) and Alzheimer's disease patients (positive group). Mathematically fitness, f(x), is defined as f(x)=FNR(x)/(FNR(x)+FPR(x)), where FNR(x) and FPR(x) are false negative and false positive rates, respectively, when x is used as a cutoff value in classification. Fitness is always a value between zero and one where zero indicates perfect similarity to the negative (reference) group and one to the positive (study) group. Relevance defines how good the test is in classifying the two diagnostic groups in consideration, defined as “sensitivity+specificity−1”. Thus, DSI is a relevance-weighted average of fitness values: DSI=sum(relevance*fitness)/sum(relevance). When differential diagnostics is performed and more than two diagnostic groups are considered, DSI is defined for each possible pair of groups and the total DSI is the average of the DSI-values. For example, if there are four diagnostics groups, Alzheimer's disease (AD), frontotemporal dementia (FTD), vascular dementia (VaD) and cognitively normal (CN), the total DSI for AD is the average of the DSI values for AD vs. FTD, AD vs. VaD and AD vs. CN. DSI is a measure that reflects the confidence of diagnosis of a test or multiple tests, i.e., the higher the DSI-value is for a disease, the more confident a specialist can be that the patient has the disease. For example, if the DSI-value for AD=0.85, FTD=0.55, VaD=0.40 and CN=0.20, it is highly probable that the patient has AD and a specialist can be relatively confident on giving diagnosis. As DSI is a generic classifier, it can be used to support many other medical decisions, not only diagnosing a patient. Many other classifiers could also be used for the same purpose instead of DSI, such as, logistic regression, random-forest, support vector machine and neural networks.

Another approach to calculate the confidence is to use data only from one patient group, instead of two or more groups, in a reference database, e.g., from healthy people, and measure whether data from a patient is atypical compared with the group, e.g. by using z-scoring. In that case, the confidence is low if the z-score is e.g. between 1^{st} and 10^{th} percentiles (z-score between −2.32 and −1.28) and otherwise high. In other words, the z-score <−2.32 would mean clearly atypical finding and >−1.28 clearly typical finding while the values between −2.32 and −1.28 would mean uncertain finding and additional testing might be justified. The use of cutoffs is discussed more below.

Further, another approach to calculate the confidence is to use probabilistic measures. Based on available test results, it is possible to use, for example the Bayesian framework to define the probability that a certain medical decision is correct, for example to define that a patient has a certain disease, or that a certain medical treatment works.

In the context of DSI, DSI values can be converted to probabilistic measures, for example by defining fitness directly as a probability or defining probability that the suggested diagnosis, i.e., the highest DSI-value over all diagnostics groups, is correct. The latter is called here as probability of correct class (PCC). PCC estimates the share of correct classifications, i.e., classification accuracy, for a given DSI-value. In the simplest form, the share of correctly classified patients in a reference database having the corresponding highest DSI value as the patient being studied may be defined. Alternatively, two highest DSI-values or any number of DSI values may be used. One possibility to define PCC is to estimate probability using the Gaussian kernel and optimize the width of the kernel using the maximum likelihood method. PCC may be defined from any measure reflecting the state of the patient, not just from DSI.

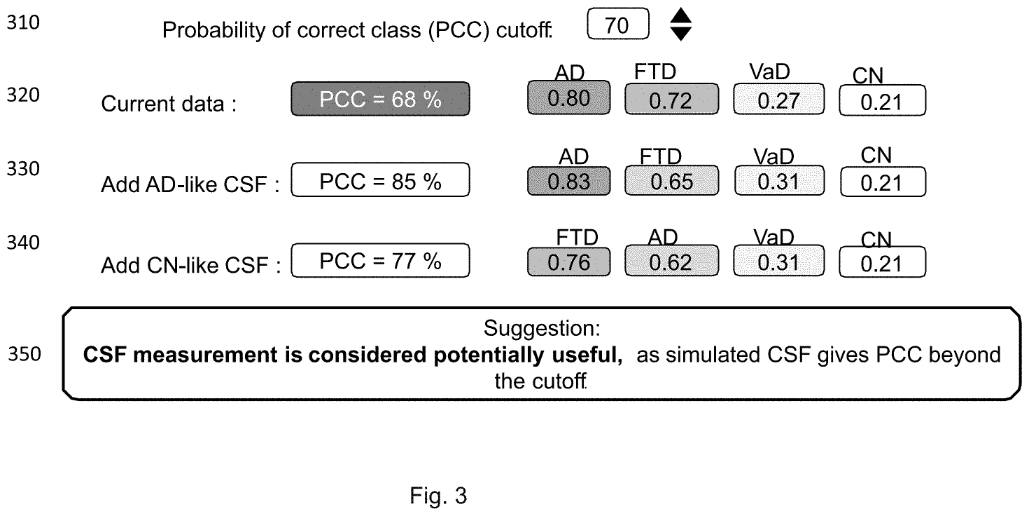

Next, a decision whether the value of confidence is high enough for making a medical decision using the existing data from a subject may be made. A cutoff value i.e. a threshold value may be applied. The cutoff value may be predetermined for the specific medical question requiring the decision. The cutoff value is defined herein for PCC. When PCC was used as the measure about confidence of medical decision, it could be defined that PCC should be, for example at least 80%, i.e., the cutoff is 80%. The optimal cutoff may depend on many factors and may be defined based on some cost-efficiency analysis. If such analysis is not available, a specialist or a hospital may define the cutoff, i.e., the minimum level of confidence for making a certain medical decision and use the value for all patients requiring the decision. In other words, it is recommended that the cutoff is fixed and not chosen for each patient separately. However, an implementation of the present disclosure could enable the user to test different cutoff values, as described later in context with FIG. 3. In the method, in step 130, when the cutoff has been chosen and the value of confidence is calculated, the first decision can be made. If the value of confidence is higher or equal than the cutoff, it can be concluded that an additional new test is not needed or justified but the medical decision could be made based on existing data, as in step 171. Herein, the value of confidence is defined to include higher or equal confidence, but it could also be defined to include only higher confidence.

Whereas, if the confidence is smaller than the cutoff, a medical decision with high confidence cannot be made and the next step 140 of the method 100 follows. Because the true value of the new further test is not known, measuring the new further test is simulated and different outcome scenarios can be tested. The steps 140-150 show the testing for one medical condition reflecting one outcome scenario but optionally these steps may be repeated for multiple conditions and scenarios as indicated by an arrow 141. One scenario could be that a patient has Alzheimer's disease and another that a patient is healthy. One could also define the outcome scenario directly from the medical condition, for example, that a patient is amyloid positive (corresponds to Alzheimer's disease) or amyloid negative (corresponds to healthy or non-Alzheimer disease). At least one value for the new further test is defined representing a certain medical condition. Values configured to be simulated for the new further test represents typical value for some medical condition and they may be received or derived, for example, from a reference database or from a medical journal. The medical condition means herein any characteristics of people that a medical test differentiates, for example a specific cognitive test measures if a patient has memory decline, a thermometer measures whether a patient has fever or hypothermia, or the concentration of amyloid beta 42 protein from CSF measures whether a patient is amyloid positive corresponding concentration values found from Alzheimer's disease patients. For example, a test of concentration of amyloid beta 42 protein from CSF may be simulated by values representing amyloid positive or amyloid negative, or a cognitive test may be simulated by values representing values of clear memory decline, less clear memory decline or no memory decline. Thus, the different simulated values of the new further test represent possible outcomes from the new further test for the patient being studied. In cognitive disorders, a specialist may consider measuring CSF biomarkers and the values simulated could represent beta-amyloid biomarker concentrations typically measured for Alzheimer's patients or healthy people. The values may be defined as a median, an average or a mode of data measured over of previously diagnosed Alzheimer's patients or healthy people in a reference database. In other words, a value could represent so called amyloid positives (patients with biomarker typical to Alzheimer's disease patients in one scenario), or amyloid negatives (patients without biomarker typical to Alzheimer's disease patients in one scenario). Multiple values for one medical condition can be simulated. Using multiple values may be a useful approach when a medical condition is composed of multiple subtypes. For example, frontotemporal dementia is a heterogeneous disease and different values could represent or be typical to different subtypes of this disease. Using multiple values may be also useful when the range of values representing a medical condition is wide and a single value does not represent them properly. On the other hand, the value of the further test may be considered as a composition of multiple measurement values. For example, if the further test that is considered to be used is CSF measurement, the simulated values could mean only amyloid beta concentrations or the concentrations of amyloid beta, total tau and phosphorylated tau biomarkers or any other combination of concentrations measured. If more than a single test result is used, the multi-dimensional simulated value may consist of, or example, median values for each test result. In other words, a value of the further test representing a medical condition, for example related to Alzheimer's disease, could then be a set of median values calculated for all relevant CSF biomarkers from previous Alzheimer's disease patients. In addition, the further test can be considered as a combination of different types of tests, for example, CSF measurements and PET imaging or some additional neuropsychological tests.

Furthermore, it is possible to take samples from the possible values of the further test following some pre-defined criteria or randomly. Also in this case, the samples contain values representative of at least one medical condition, they may be, for example values corresponding healthy people or unhealthy people, as otherwise the use of the test for making a medical decision becomes difficult. As there exist multiple ways to define the representative values of each medical condition, the descriptions above should be interpreted only as examples of possible implementations.

Because the values of the further test may depend on the patient's background factors, such as age or sex of the patient, one or several such background factors may be taken account when defining the representative, i.e., typical, values of different medical conditions. Correspondingly, the existing medical test data could be utilized in simulating a representative value for the further test when the patient is expected to have a certain medical condition. There are multiple methods to implement this, for example linear regression may be used to correct values for background factors and/or existing medical test values, and remove the effect of these covariates from the values of the further test, or one could estimate most probable values using a probabilistic model which estimates the value of the further test when background factors and/or existing medical test values are given.

The simulation may be limited only to one or two medical conditions, but it is also possible that a further test may have representative values from multiple conditions, for example representing multiple diseases. In other words, there may be multiple different values for each medical condition and/or there may be multiple medical conditions. The use of more than one medical condition means that the steps 140-150 may be repeated multiple times, once for each outcome scenario.

In the next step 150, the value of confidence of medical decision is recalculated but the existing data of a subject are augmented with simulated data i.e. selected values from the further test. The data of a subject comprising measured medical test data and possibly background factors of a patient (subject) are augmented with each simulated value from the further test indicative on a medical condition producing the confidence measure for each simulated value separately. If more than one value is simulated for a medical condition, the combined confidence of medical decision linked to this condition can be defined, for example, as the maximum value or an average value of all confidence values. If averaging is used, single confidence values could be weighted based on how typical the simulated values are meaning that atypical rare values do not get much weight. Many other strategies are also possible for defining the confidence value combining single confidence values from different simulated values.

In cognitive disorders, if existing data consist of MMSE, CERAD and the hippocampus volume (HCV), and the simulated value represent amyloid positive or amyloid negative CSF biomarkers, representing two medical conditions and two output scenarios, the measure about confidence is estimated using MMSE, CERAD, HCV and amyloid positive CSF test result values or using MMSE, CERAD, HCV and amyloid negative CSF test result values. From these test results, MMSE, CERAD and HCV are true values measured from the patient while the concentrations of amyloid beta CSF biomarker values are simulated values. For the sake of clarity, if the further test produces multi-dimensional output, for example, amyloid-beta, total tau and phosphorylated tau biomarkers from CSF, the simulated value can be interpreted to be a value from only one dimension, e.g., concentration of amyloid beta biomarker, or be multi-dimensional, e.g. concentrations of amyloid beta, total tau and phosphorylated tau biomarkers.

In step 160, the values of confidence produced by each simulated scenario (medical condition) is compared with a cutoff. The cutoff used in step 160 does not need to be the same as in step 130 but there is often no reason why it should be different. If any of the tested scenarios (medical conditions) gives a value of confidence of medical decision higher or equal than the cutoff, it means that the further test may be potentially useful and may lead to a medical decision. In step 170, information about the new further test that may ease the medical decision making may be indicated, for example by a message or certain color. If none of the scenarios compared in step 160 produces a value of confidence exceeding the cutoff, the further test can be considered to not be useful. This may be indicated in step 171.

If the scenarios tested covered all possible values of the further test and none produced high value of confidence, a specialist could be certain that the further test would not produce useful results for the patient in question. However, if all, even very unlikely values, were simulated, the likelihood of getting high confidence for one scenario would increase considerably in this case which might lead to a situation where the further test is recommended for all patients and costs would increase dramatically. As already discussed, related to 150, one solution could be to weight simulated values differently based on how typical they are when defining a combined confidence value related to one medical condition. On the other hand, if only median values representing two medical conditions are used, the true measured value of the patient from the further test might be such that confidence would be high enough for medical decision although none of the scenarios tested suggested that. Opposite could happen as well when all scenarios tested may show high confidence but the true measured value of the further test not. This can happen, for example if the true measured value of the further test is in the middle of the two simulated values.

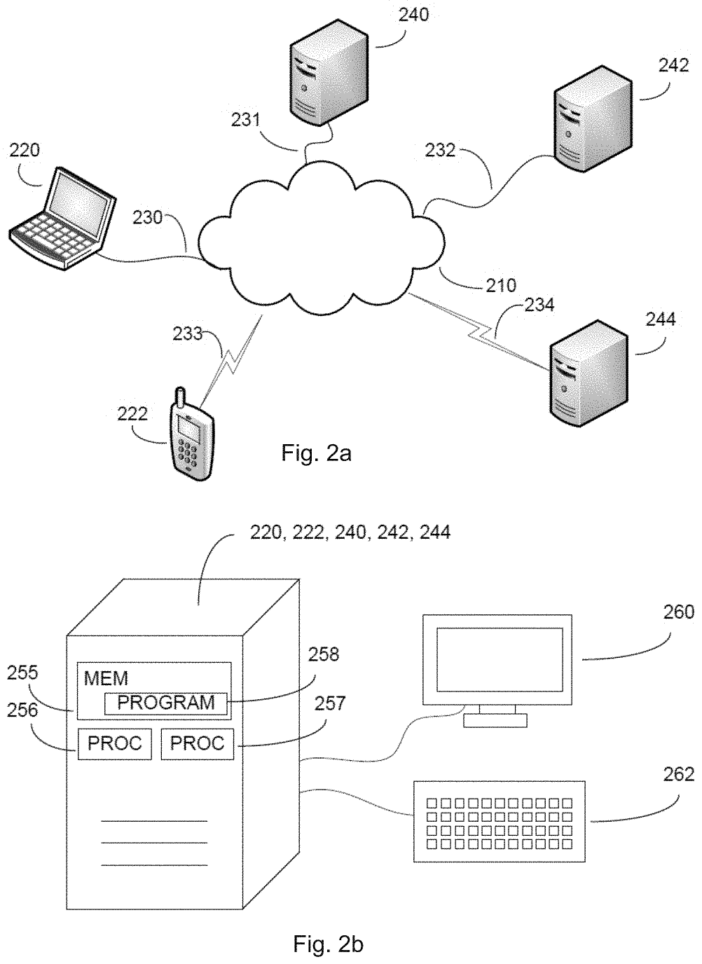

FIG. 2a shows, by way of example, devices and a system arranged to infer the justification of a further medical test. The different devices are connected via a network 210 such as the Internet or a local area network or any wired or wireless communication network. There are a number of servers connected to the network 210, and here are shown a server 240 for offering a network service, for example for classifying a system, a server 242 for storing datasets related to the service and a server 244 for processing data and performing computations. These servers may be made of multiple parts or they may be combined into one more servers.

There are also a number of end-user devices such as personal computers 220 and mobile phones 222. These devices 220 and 222 may also be made of multiple parts. The various devices are connected to the network 210 via communication connections such as a fixed connection 230, 231 and 232 or a wireless connection 233 and 234. The connections may be implemented by means of communication interfaces at the respective ends of the communication connection.

FIG. 2b shows, by way of example, a device arranged to infer the justification of a further medical test. The device 220, 222, 240, 242 or 244 contains memory 255, one or more processors 256, 257, and computer program code 258 residing in the memory 255 for implementing, for example, computations for inferring the justification of a further medical test. The device may also be functionally connected to a display 260 for example for displaying different confidence values of medical decision or message indicating whether a new further medical test would be beneficial in medical decision making. There may also be various input means functionally connected to the device, such as a keyboard 262, speech command interface, data gloves, and different communication interfaces for receiving input (not shown).

FIG. 3 shows, by way of example, a representation and visualization of confidence of medical decision when only existing data are used and when two scenarios, i.e., whether a patient has CSF measurements similar to Alzheimer's disease or cognitively normal, are used. This implementation example supports a user in deciding whether CSF biomarkers should be measured when diagnosing cognitive disorders. Row 310 indicates the chosen cutoff value of confidence of medical decision. The PCC-cutoff can be predetermined, for example for a certain disease or a medical decision, for example by a hospital or an insurance company. In FIG. 3, the example shows an implementation where the user can test the impact of different PCC-cutoffs for the result. The “Current data” row 320 shows the DSI-values for four diagnostics groups AD=0.80, FTD=0.72, VaD=0.27 and CN=0.21 using the existing and currently available data from already performed medical tests, which in this example are cognitive tests and MRI imaging. With these DSI-values, the value of confidence, measured by PCC, is 68% which is below the current PCC-cutoff 70%. This indicates that the confidence may not be high enough to make a diagnosis. Therefore, the user may consider whether a new further medical test is needed and justified, which in this example is measuring of CSF. The user considers that this further test of measuring CSF may possibly increase the value of confidence i.e. the PCC value by testing two scenarios. The “Add AD-like CSF” row 330 shows the DSI-values and the current value of confidence PCC when the existing patient data are augmented with the median CSF biomarker-values from AD patients. In other words, this row 330 shows a scenario when the true CSF biomarker values for this patient would be AD-like, simulated by the median values from AD patients corresponding to the age and gender of the patient. If the patient had such AD-like CSF-biomarker values, the AD diagnosis would be correct in 85% probability. On the other hand, the “Add CN-line CSF” row 340 shows correspondingly the DSI-values and their value of confidence, measured by PCC, when the existing patient data are augmented with the median CSF-values from CN people corresponding to the age and gender of the patient. If the patient has such CN-like CSF-biomarker values, the FTD diagnosis is correct in 77% probability. These results of rows 330 and 340 show that in the both scenarios, PCC becomes higher than the cutoff (85% □ 70% and 77% □ 70%) and CSF measurement could be considered potentially useful, as visualized to the user in result row 350 as a message. The corresponding message would be obtained even if only either of the simulated scenarios produced PCC □ 70%.

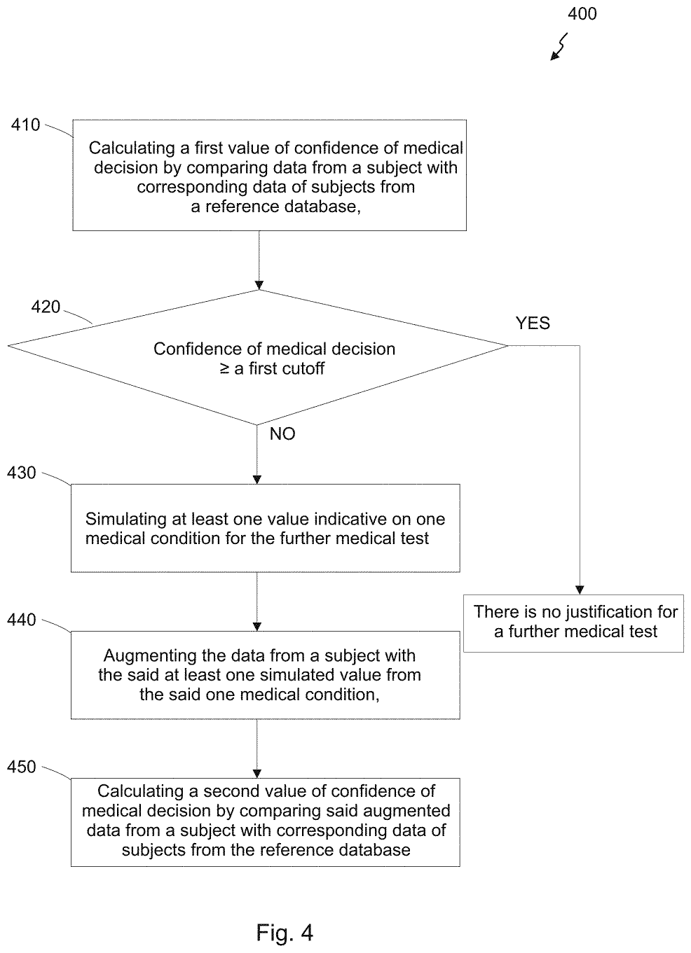

FIG. 4 shows, by way of example, a method 400 for inferring a justification for performing a further medical test. In step 410, a first value of confidence of medical decision is calculated by comparing data from a subject with corresponding data of subjects from a reference database. Data from a subject may be obtained, for example, received from a database or a patient folder. The data from a subject configured to be used for inferring a justification for a further medical test may comprise, for example, data from medical test, for example cognitive tests or magnetic resonance imaging and/or background factors of a patient i.e. subject, for example data of age, gender, number of education years, co-morbidities, and medications used. And if the first value of confidence of medical decision, in step 420, is smaller than a first cutoff, which defines the minimum acceptable confidence value for making a medical decision, the following steps are performed: In step 430, at least one value indicative on one medical condition for the further medical test is defined by simulating, in step 430, one or more values indicative on one medical condition for the further medical test. The one or more values represent typical values for said medical condition, for example values of healthy person or alternatively values of un-healthy person. The simulation may be repeated for multiple medical conditions. In step 440, the data from a subject is augmented with said at least one simulated value from said one medical condition. And in step 450, a second value of confidence of medical decision is calculated by comparing said augmented data from a subject with corresponding data of subjects from the reference database i.e. using data from a subject and at least one simulated value.

Thus, in the method of inferring a justification for a further medical test of the present disclosure, if a medical decision (e.g. diagnosis of disease) cannot be made based on data from a subject comprising data of medical test(s) that has(have) already been performed for a patient/subject i.e. actual data, medical test data i.e. value(s) received/obtained from a reference database is used in addition to actual data from a subject for whom it is being considered whether a further test is justified. The medical test data received from the reference database relates to the medical condition and is data of a test that is considered to be performed for the patient/subject i.e. result value(s) of the medical test when it was earlier performed for other patient(s) or subject(s) who are in that medical condition in order to make the medical decision determination easier. Then, actual data from a subject and reference database data of other subject(s) are both used when a value of confidence of making a medical decision is calculated. And if the value of confidence is at least as high as predetermined cutoff value of confidence (measured e.g., by PCC) then doing a further test for the patient is justified and making the medical decision for the patient should be easier.

It should be noted that in some example embodiments, some of the method steps may not need to be performed, number of performed medical tests may vary as well as number and type of used values of medical tests received from a reference database.

The various embodiments of the present disclosure may be implemented with the help of computer program code that resides in a memory and causes the relevant apparatuses to carry out the present disclosure. For example, a personal computer may comprise circuitry and electronics for handling, receiving, and transmitting data, computer program code in a memory, and a processor that, when running the computer program code, causes the computer to carry out the features of an example embodiment. Yet further, a server may comprise circuitry and electronics for handling, receiving, and transmitting data, computer program code in a memory, and a processor that, when running the computer program code, causes the server to carry out the features of an example embodiment.

It is obvious that the present disclosure is not limited solely to the above-presented examples, but it can be modified within the scope of the appended claims.

1. A method for inferring a justification for a further medical test, comprising:
calculating a first value of confidence of medical decision by comparing data from a subject with corresponding data of subjects from a reference database,
if the first value of confidence of medical decision is smaller than a first cutoff, which defines the minimum acceptable confidence value for making a medical decision, the following steps are performed:
simulating at least one value indicative on one medical condition for the further medical test, wherein said at least one value represents typical value for said medical condition, and
augmenting the data from a subject with said at least one simulated value from said one medical condition, and
calculating a second value of confidence of medical decision by comparing said augmented data from a subject with corresponding data of subjects from the reference database.

2. A method according to claim 1, wherein the method further comprises
indicating that the further medical test is justified if said second value of confidence of medical decision is higher than a second cutoff.

3. A method according to claim 1, wherein a value of confidence of medical decision is defined using a probabilistic measure.

4. A method according to claim 1, wherein a value of confidence of medical decision is defined using a probability of correct class (PCC).

5. A method according to claim 1, wherein a value of confidence of medical decision is defined using disease-state index, which measures the location of said data from a subject relative to two groups of subjects.

6. A method according to claim 1, wherein said data from a subject comprises medical test data comprises data from cognitive tests or magnetic resonance imaging.

7. A method according to claim 1, wherein said data from a subject comprises background factors of the subject.

8. A method according to claim 7, wherein background factors of the subject comprise age, gender, number of education years, information about co-morbidities, or medications used.

9. A method according to claim 7, wherein said at least one simulated value of the further medical test is corrected for at least one background factor of the subject.

10. A method according to claim 6, wherein said at least one simulated value of the further medical test is corrected for medical test data of the subject.

11. A method according to claim 1, wherein the further medical test is cerebrospinal fluid biomarkers or positron emission tomography imaging.

12. A method according to claim 1, wherein said confidence of medical decision is confidence of giving a certain treatment.

13. A method according to claim 1, wherein said confidence of medical decision is confidence of diagnosis.

14. A method according to claim 1, wherein said confidence of medical decision is confidence of diagnosis in cognitive disorders.

15. A method according to claim 1, wherein the first cutoff is the same as the second cutoff.

16. A computer program product embodied on a non-transitory computer readable medium, the computer program product comprising computer instructions that, when executed on at least one processor of a system or an apparatus, is configured to perform the method according to claim 1.

17. A device for inferring a justification for a further medical test comprising means for performing the method according to claim 1.
